(19) United States
(12) Patent Application Publication
(10) Pub. No.: US 2009/0214475 A1
(43) Pub. Date: Aug. 27, 2009

(54) Extractability and Bioavailability of the Natural Antioxidant Astaxanthin From a Green Alga, Haematococcus Pluvialis

(75) Inventors: Qiang Hu, Chandler, AZ (US); Milton R. Sommerfeld, Chandler, AZ (US); Fan Fu, Clemmons, NC (US)

Correspondence Address: FULBRIGHT & JAWORSKI L.L.P., 600 CONGRESS AVE., SUITE 2400, AUSTIN, TX, 78701, US

(73) Assignees: ALGAEN CORPORATION, Winston-Salem, NC (US); ARIZONA BOARD OF REGENTS FOR AND ON BEHALF OF ARIZONA STATE UNIVERSITY, Scottsdale, AZ (US)

(21) Appl. No.: 11/887,592

(22) PCT Filed: Mar. 31, 2006
(86) PCT No.: PCT/US2006/011826

§ 371 Date: May 6, 2009

Related U.S. Application Data
(60) Provisional application No. 60/666,855, filed on Apr. 1, 2005.

(51) Int. Cl. A61K 35/66; C12P 7/26; C12N 1/00; C12N 1/12; A23L 1/28

(52) U.S. Cl. 424/93.1; 435/148; 435/317.1; 435/257.6; 426/61

(57) ABSTRACT

As the richest source of astaxanthin, a natural antioxidant and coloring agent, the unicellular green alga, Haematococcus pluvialis, is being commercially exploited. A major constraint in the Haematococcus production system, however, is the thick, rigid cell walls associated with astaxanthin-rich cysts (or aplanospores). The thick walls prevent the extraction of cellular materials and consequently reduce the bioavailability of astaxanthin. Using a physical, chemical, or enzymatic method to disrupt the cell wall has proven to be very expensive and also introduce the risk of oxidation of astaxanthin by atmospheric oxygen The present invention provides a novel method for solving this problem by introducing two genetically modified Haematococcus pluvialis mutants. These two mutants, named as D 13-17 and N54-22, contain remarkably reduced amounts of cell wall materials, but retain the growth potential and ability to accumulate astaxanthin as high as the wild type strain. Organic solvent extraction efficiency assay has demonstrated that cellular astaxanthin can be more effectively and efficiently extracted from the cell wall-deficient mutants than from the wild type, suggesting that the mutants may provide better bioavailability of astaxanthin to humans and animals. The said mutants can be used for production of natural astaxanthin for human and animal consumption

### STATEMENT OF RELATED APPLICATIONS

The present application claims priority under 35 USC §119 provisional application 60/666,855 filed Apr. 1, 2005, the disclosure of the provisional application being hereby incorporated by reference.

### FIELD OF THE INVENTION

The present invention relates to a method of improving the extractability and bioavailability of natural astaxanthin using two mutant strains of Haematococcus pluvialis; methods for generation, selection and characterization of the said mutants; and their use in animal feed, human dietary supplements, pharmaceuticals, foods and the like.

### BACKGROUND OF THE INVENTION

The natural red pigment astaxanthin (3,3′-dihydroxy-4,4′-dione-β,β′-carotene) is a potent bioactive antioxidant that offers potential for applications in nutraceutical and pharmaceutical industries (Guerin et al., 2003). Astaxanthin is also being widely used in aquaculture and poultry industries as a feed additive to improve the coloration of cultured salmons, crustaceans, and egg yolks (Borowitzka, 1997; Lorenz and Cysewski 2000).

The unicellular green alga, Haematococcus pluvialis, is the richest known natural source of astaxanthin, with its cellular content of the pigment reaching as high as 4% of the cell dry weight under certain stress conditions (Lu et al., 1994; Boussiba et al., 1999). In recent years, mass production of H. pluvialis has attracted considerable biotechnological attention worldwide, and Haematococcus-derived astaxanthin has become commercially available (Lorenz and Cysewski 2000). Natural astaxanthin production and commercialization is estimated to be a 1.2 billion dollar annual market.

A major constraint in the Haematococcus production system is, however, that the astaxanthin-rich cells (cysts or aplanospores) possess thick cell walls that impair the extraction of the cellular astaxanthin and consequently reduce the bioavailability of astaxanthin for human and animal consumers (Castenmiller and West 1997; Mendes-Pinto et al., 2001). Bioavailability can be defined as the proportion of a nutrient ingested which becomes available to the body for metabolic processes or the proportion of a nutrient that is capable of being absorbed and available for use or storage (Castenmiller and West 1997). Indeed, very poor pigmentation in salmonids and other cultured animals were often observed when fed with intact Haematococcus biomass, and considerable enhancement of astaxanthin deposition in these animals was obtained with a diet containing disrupted or broken astaxanthin-rich Haematococcus cysts (Good and Chapman, 1979; Sommer, et al. 1991; Choubert and Heinrich, 1993).

Various physical and chemical processes, such as high-pressure homogenization, ball milling, autoclaving, enzymatic digestion, and pH-dependent hydrolysis, have been used to promote the disruption of the thick-walled cysts (Nonomura, 1987; Bubrick, 1991; Mendes-Pinto et al., 2001). However, these processes are either inefficient or expensive. Up to 25% of the production costs have been reported to be spent on cell disruption/breakage processes. In addition, these methods introduce the risk of oxidation and degradation of astaxanthin by atmospheric oxygen, and thus artificial antioxidants of various kinds may have to be provided to prevent pigment oxidation (Bubrick, 1991).

### BRIEF SUMMARY OF THE INVENTION

The present invention provides a novel concept to improve the extractability and bioavailability of astaxanthin from H. pluvialis. By using a conventional mutagenesis method, two cell wall-deficient mutants of H. pluvialis that contain only residual amounts of cell wall materials were generated, and therefore allowing the improved extraction bioavailability of astaxanthin, but retain the growth potential and ability to accumulate astaxanthin at a level comparable to the wild type strain (WT).

### BRIEF DESCRIPTION OF THE TABLES AND DRAWINGS

Table 1. Cellular contents of astaxanthin in wild type and putative wall deficient mutants.

Table 2. HPLC-separation and identification of carotenoids in various strains of H. pluvialis.

Table 3. Efficiency of pigment extraction from Haematococcus strains by ethanol, methanol, chloroform and DMSO

FIG. 1 shows the Effects of MNNG treatment time (A) and dose (B) on cell survival rate of H. pluvialis.

FIG. 2. Light micrographs of red cysts of the wild type (A, D) and representative cell wall-deficient mutants (B, C, E, F) of H. pluvialis.

FIG. 3. The specific growth rates of putative cell wall deficient mutants relative to the wild type of H. pluvialis.

FIG. 4. Cell recovery potential among various Haematococcus strains after being treated with 1% Triton X-100 for 10 min.

FIG. 5. HPLC profiles of carotenoids and chlorophylls extracted from the WT (A), mutants D13-17 (B) and N54-22 (C) of H. pluvialis.

FIG. 6. Electron micrographs of thin sections of the wild type (A), mutants D13-17 (B) and N54-22 (C)

### DETAILED DESCRIPTION OF THE INVENTION

### 1). Effects Of MNNG Dose And Treatment Time On The Survival Rate Of Haematococcus

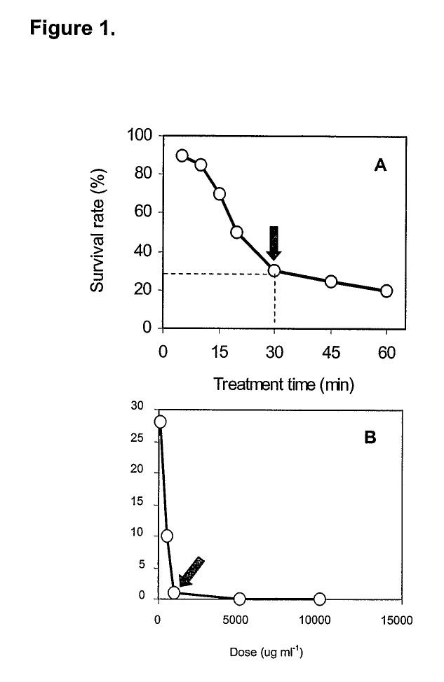

Cell suspension at a concentration of 2×10⁵ cells mL⁻¹ were treated with 100 μg mL⁻¹ MNNG for 5, 10, 15, 20, 30, 45, and 60 min, respectively. Formation of mutant colonies, if any, occurred on agar plates two weeks after MNNG treatment, and cell survival rates were calculated accordingly. As shown in FIG. 1A, treating cells with 100 μg mL⁻¹ MNNG for 5 or 10 min did not cause significant cell death. Considerable cell death occurred in the 15 min treatment group, resulting in ca. 70% survival rate. Dramatic decrease in survival rate (<30%) was observed in 30-min treatment group. Further increase in treatment time to 60 min did not result in proportional decrease in survival rate, and the rate was around 20%. As such, a 30-min time frame for mutagenesis treatment was chosen for mutagen dosage assessment.

In order to achieve low survival rates of 1˜5%, or high mortality (over 95% or even 99%), as suggested by Carlton and Brown 1981, for obtaining microbial mutants with significant changes in cellular structure and function, we investigated the effect of MNNG concentration on Haematococcus survival rate. MNNG concentrations were 100, 500, 1,000, 5,000, and 10,000 μg mL⁻and treatment time was 30 min. As shown in FIG. 1B, treatment with 1,000 μg MNNG mL⁻resulted in a suitable survival rate of ca. 1%. The concentrations of MNNG above this level resulted in 0.01 to 0.001%, which might be too low for our purposes. Using the improved mutagenesis protocol, which was to treat cells with 1,000 μg MNNG mL⁻¹ for 30 min, we have obtained 5,127 mutated colonies.

### 2) Screening of Putative Cell Wall Mutants: Light Microscopy

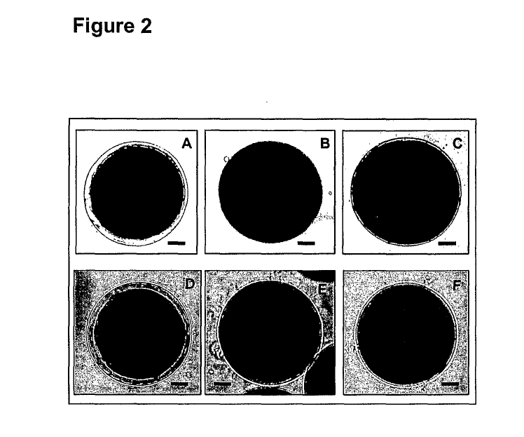

The 5,127 mutated colonies were obtained and subjected to initial screening using light microscopy. Because mature red cysts of H. pluvialis were large (i.e., 30˜50 μm in diameter) with thick cell walls ranging from 2 to 4 μm and exhibited a broad, relatively transparent space filled with gelatinous matrix between the cell wall and the protoplast, any major alteration in wall thickness and morphology could be observed with light microscopy. FIGS. 2A & D are micrographs of wild type red cysts featuring the thick wall and distinct intervening space. In contrast, FIGS. 2B, C, E, F are examples of wall-deficient mutants. Of 5,127 subcultures of the mutagenized colonies, 37 mutants with altered wall morphology were identified.

### 3). Physiological And Biochemical Characterization Of Cell Wall-Deficient Mutants

3.1) Specific Growth Rates

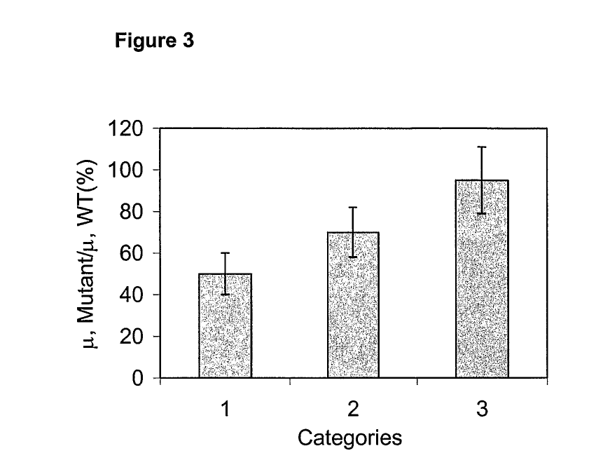

The specific growth rate, which affects potential biomass/astaxanthin production in a large-scale photobioreactor, is an important criterion for selection of a cell wall deficient mutant. All 37 putative cell wall deficient mutants were subjected to growth analysis. Roughly, these mutants fell into three major categories based upon the specific growth rate: 18 cell wall deficient mutants had extremely low specific growth rates, 12 mutants showed reduced specific growth relative to the wild type, and 7 mutants exhibited the specific growth rates similar to the wild type (FIG. 3). This suggests that most of the wall deficient mutants obtained from this study might have had significant genetic mutations not only occurring in cell wall biosynthesis but also exerting effects on other aspects of cellular metabolism, resulting in a decreased specific growth rate.

3.2) Susceptibility Of Cell Wall Deficient Mutants To Detergent

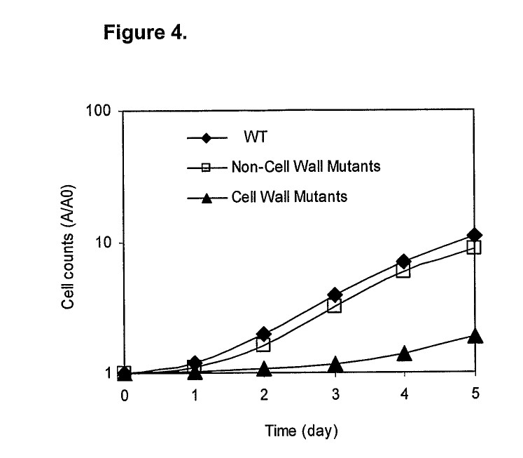

The integrity of the cell wall of the wall deficient mutants were further evaluated by incubating individual mutants with 1% Triton X-100 for 10 min at 25° C. For comparison, the same treatments were also applied to non cell wall-deficient mutants and wild type. After detergent treatment, cells were washed three times with dH₂O and re-suspended in growth medium at a final concentration of 2×10⁵ cells ml⁻and incubated in flasks under a light intensity of 20 μmol m⁻² s⁻¹. Cell numbers were counted daily to determine the viability of treated cells and their ability to recover. As shown in FIG. 4, the growth rates of wild type and non-cell wall mutants were high and similar. In contrast, most of the cell wall-deficient mutants experienced a longer lag phase and exhibited considerably lower growth potential compared to the wild type. These results indicate that the putative cell-wall deficient mutants were indeed impaired (reduced cell wall thickness and/or altered structure) and thus are more susceptible to detergent treatment.

3.3) Pigment Composition

The wild type strains of Haematococcus pluvialis can accumulate 2˜3% astaxanthin on a per dry weight basis under various stress conditions. Seven mutants that exhibited the growth potential comparable to the wild type were subjected to cellular astaxanthin content analysis and results are shown in Table 1. Of seven mutant strains, four strains have the ability to accumulate astaxanthin concentrations as high as the wild type. One mutant strain can accumulate ⅔ of that found in the wild type. Yet, two mutant strains had the pigment contents less than ⅙ of the wild type. Clearly, the latter group of the mutants had impaired pigment biosynthetic pathway, resulting in significant reduction in the astaxanthin production.

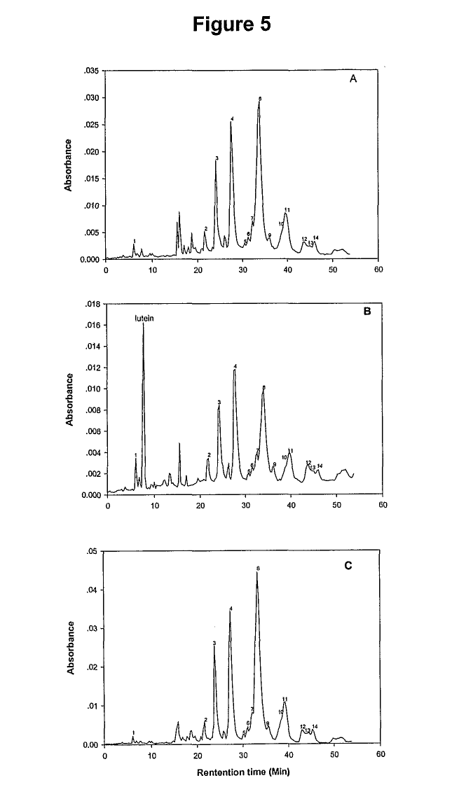

FIG. 5 shows the typical HPLC chromatograms of pigments extracted from the wild type and mutants, D13-17 and N54-22. Identification of individual pigment species isolated in FIG. 5 was shown in Table 2. As expected, various esterified forms of astaxanthin were present as the major carotenoids in red cysts of the wild type (FIG. 5A). Astaxanthin esters accounted for 90.8% of total carotenoids, corresponding to 21.65 mg per gram of cell dry weight. These results were consistent with those reported using the same algal strain (Kobayashi et al., 1991 and 1993; Steinbrenner and Linden, 2001). The chromatograms of D13-17 and N54-22 resembled that of the wild type, although a notable decrease in astaxanthin but increase in lutein occurred in D13-17 (FIGS. 5B, C).

3.4) Ultrastructure Of The Cell Wall

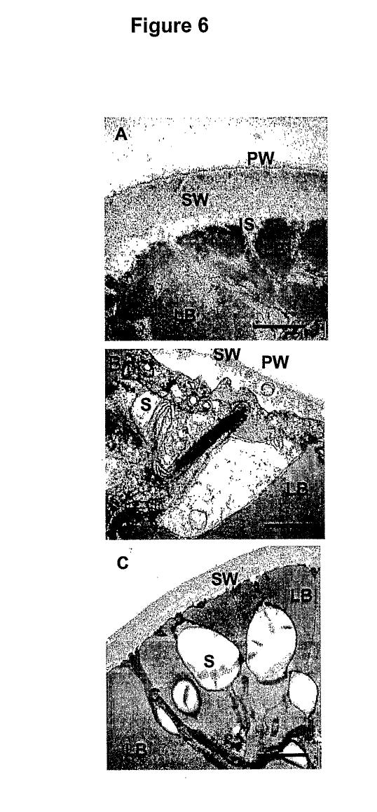

Ultrastructure of the cell wall was examined for both wild type and the mutants by transmission electron microscopy. Although the formation of lipid bodies occurred in both the wild type and mutant cysts under stress, cell wall structure of the mutants differed considerably from the wild type. A thick secondary wall surrounded by the remains of a trilaminar sheath and the primary wall was seen in a wild type cyst. The intervening space filled with a gelatinous matrix was also evident (FIG. 6A). In contrast, the thickness of the secondary cell wall was reduced in D13-17 and contained granular inclusions, presumably containing wall precursors, in the intervening space (FIG. 6B). Compared to the wild type, N54-22 also had a reduced secondary cell wall with little intervening space present between the cell wall and plasma membrane (FIG. 6C).

### 4) Bioavailability: Pigment Extraction Efficiency Assay

Extraction of total pigments from algal cells with an organic solvent may provide a simple, rapid estimation of the extent to which the cell wall is defective, assuming that mutants with impaired/reduced cell walls might make the cytoplasmic membrane more susceptible to organic solvent/s.

The seven mutants that had the growth rates similar to the wild type were subjected to evaluation by the pigment extraction efficiency assay. Four mutants showed considerably higher pigment extraction efficiencies compared to wild type and the other three mutants tested (Table 3). Extraction efficiencies of total pigments from the four mutants were over 80% using DMSO, whereas that of the wild type and the other three mutants was less than 30%. This is an exciting result because it indicates that application of the cell wall-deficient mutants may result in improved bioavailability of cellular astaxanthin to humans and other animals.

### REFERENCES CITED

Borowitzka, M. A. 1997. J. Appl. Phycol. 9:393-401.
Boussiba, S., Wang, B., Yuan, J. P., Zarka, A., and Chen, F. 1999. Biotechnol. Lett. 21:601-604
Bubrick, P. 1991. Bioresource Technology 38: 237-239
Carlton B. C. and Brown B. J. (1981) in Manual of methods for general microbiology, chapter 13, pp. 22-42, Washington, D.C.: American Society for Microbiology.
Castenmiller, J. J. M. and West, C. E. 1997. Pure Appl. Chem. 69:2145-1250
Choubert, G. and Heinrich, O. 1993. Aquaculture 112: 127-226
Good, B. H., and Chapman, R. L. 1979. J. Phycol. 15: 17
Guerin, M., Huntley, M. E., and Olaizola, M. 2003. Trends Biotechnol. 21: 210-216
Kobayashi, M., Kakizono, T., and Nagai, S. 1991. J Ferment Bioeng 71:335-339.
Kobayashi, M., Kakizono, T., and Nagai, S. 1993. Appl. Environ. Microbiol. 59:867-873
Lorenz, R. T., and Cysewski, G. R. 2000. Trends Biotech. 18:160-167
Lu, F., Vonshak, A., and Boussiba, S. 1994. J. Phycol. 30:829-833.
Mendes-Pinto, M. M., Raposo, M., F., J., Bowen, J., Young, A. J., and Morais, R. 2001. J. Appl. Phycol. 13:19-24
Nonomura, A. M. 1987. U.S. Pat. No. 4,680,413
Rosenfeld J, Capdevielle J, Guillemot J C, Ferrara P. 1992. Anal Biochem. 15;203(1):173-9.
Sommer, T. R., Potts, W. T., and Morrisy, N. M. 1991. Aquaculture 94: 79-88
Steinbrenner, J., and Linden, H. 2001. Plant Physiol. 125:810-817
Voigt J, Frank R, 2003. Plant Cell. 15(6): 1399-413.
Yuan, J. P., Chen, F. Liu, X., and Li X. Z. 2002. Food Chem. 76:319-325.

TABLE 1
Cellular contents of astaxanthin in wild
type and putative wall deficient mutants

| | Astaxanthin |
|---|---|
| Strain Name | (% of dry weight) |
| Wild Type | 3.2% |
| Cell-Wall Mutants | |
| D13-17 | 3.3% |
| N54-22 | 3.2% |
| N29-32 | 3.1% |
| D36-29 | 3.3% |
| N44-6 | 2.3% |
| D27-38 | 0.5% |
| N0656 | 0.4% |

TABLE 2
HPLC-separation and identification of carotenoids in various strains
of H. pluvialis. Samples were taken 5 days after onset of stress.

| Peak | Retention | Absorbance | |
|---|---|---|---|
| Number | time (min) | maxima (nm) | Pigments |
| 1 | 6.0 | 480.0 | (3S, 3′S)-trans-astaxanthin |
| 2 | 21.5 | 482.5 | (3S, 3′S)-trans-astaxanthin ester |
| 3 | 24.0 | 482.5 | (3S, 3′S)-trans-astaxanthin ester |
| 4 | 27.5 | 482.5 | (3S, 3′S)-trans-astaxanthin ester |
| 5 | 31.3 | 482.5 | (3S, 3′S)-trans-astaxanthin ester |
| 6 | 32.2 | 472.8 | (3S, 3′S)-9-cis-astaxanthin ester |
| 7 | 33.2 | 472.8 | (3S, 3′S)-13-cis-astaxanthin ester |
| 8 | 33.7 | 482.5 | (3S, 3′S)-trans-astaxanthin ester |
| 9 | 35.8 | 482.5 | (3R, 3′R)-trans-astaxanthin ester |
| 10 | 38.8 | 472.8 | (3S, 3′S)-9-cis-astaxanthin ester |
| 11 | 39.4 | 472.8 | (3S, 3′S)-9-cis-astaxanthin ester |
| 12 | 43.7 | 482.5 | (3S, 3′S)-trans-astaxanthin ester |
| 13 | 44.9 | 482.5 | (3R, 3′R)-trans-astaxanthin ester |
| 14 | 45.9 | 482.5 | (3R, 3′R)-trans-astaxanthin ester |

TABLE 3
Efficiency of pigment extraction from Haematococcus
strains by ethanol, methanol, chloroform and DMSO.

| | | | | | E: DMSO |
|---|---|---|---|---|---|
| Strain Name | A: Ethanol | B: Methanol | C: Chloroform | D: DMSO | Control |
| Wild Type | 7% | 5% | 8% | 8% | 100% |
| Cell-Wall Mutants | | | | | |
| D13-17 | 40% | 45% | 50% | 87% | 100% |
| N54-22 | 38% | 44% | 47% | 85% | 100% |
| N29-38 | 41% | 47% | 49% | 79% | 100% |
| D36-56 | 41% | 44% | 51% | 89% | 100% |
| Average | 40% | 45% | 49% | 85% | 100% |
| N44-6 | 25% | 27% | 29% | 32% | 100% |
| D27-32 | 28% | 23% | 24% | 25% | 100% |
| N06-29 | 26% | 24% | 25% | 30% | 100% |
| Average | 26.3% | 24.7% | 26% | 29% | 100% |

We claim:

1. A method for improving the extractability and bioavailability of natural astaxanthin from astaxanthin-containing organisms, including but not limited to Haematococcus pluvialis, comprising:
a. Generating mutants of the astaxanthin-containing organisms, including but not limited to Haematococcus pluvialis that have deficient or significantly reduced cell wall by chemical or physical means.
b. selecting the said mutants of the said organisms, including but not limited to Haematococcus pluvialis.
c. characterizing the said mutants of the said organisms, including but not limited to Haematococcus pluvialis.

2. According to claim 1, the said mutants of Haematococcus pluvialis are: D13-17 and N54-22.

3. According to claim 1, the said mutants of the said organisms, including but not limited to Haematococcus pluvialis, contains deficient or significantly reduced cell wall.

4. According to claim 1, the methods for generating the said mutants include chemical mutagenesis by using MNNG and physical means of exposing the cells of the said organism to UV light.

5. According to claim 1, the methods for selecting the said mutants include light microscope and electron microscope.

6. According to claim 1, the methods to characterize the said mutants include measurements on specific growth rate and pigment composition and concentration; test the susceptibility of the said mutants to detergents; test the extractability of the pigments from the said mutants.

7. A composition comprising the biomass of the said mutants of the said organisms, or cellular compositions, including but not limited to pigments, lipids, proteins, vitamins, mineral nutrients, extracted from the said mutants of the said organisms, including but not limited to Haematococcus pluvialis.

8. According to claim 7, the said biomass is the whole cells of the said mutants of the said organisms, including but not limited to Haematococcus pluvialis, in fresh form or dried form.

9. According to claim 7, the said cellular compositions include all contents from a cell of the said mutants of the said organisms, including but not limited to Haematococcus pluvialis.

10. According to claim 7, the said composition further comprises a medicine for treating inflammatory diseases, cardiovascular diseases and cancers.

11. A food product comprising the said composition according to claim 7.

12. The food product according to claim 11 wherein astaxanthin is present in an amount that is at least 5 mg.

13. A dietary supplement product comprising the said composition according to claim 7.

14. The dietary supplement product of claim 13, wherein the supplement is in the form of a capsule or tablet.

15. A beverage product comprising the said composition according to claim 7.

16. An animal feed comprising the said composition according to claim 7.

17. The said animal feed according to claim 16 include all feeds for aquaculture, poultry, pork, beef and dairy industries.
